(19) United States
(12) Patent Application Publication
(10) Pub. No.: US 2008/0084541 A1
(43) Pub. Date: Apr. 10, 2008

(54) Ophthalmic system and method

(76) Inventors: Ming Lai, Webster, NY (US); Barry T. Eagan, Spencerport, NY (US); Casey Stack, Bountiful, UT (US); Joseph Bentley, West Jordan, UT (US); Daozhi Wang, Rochester, NY (US)

Correspondence Address: Bausch & Lomb Incorporated, One Bausch & Lomb Place, Rochester, NY, 14604-2701, US

(21) Appl. No.: 11/544,541

(22) Filed: Oct. 6, 2006

(51) Int. Cl. A61B 3/10

(52) U.S. Cl. 351/221

(57) ABSTRACT

Ophthalmic system and method particularly suited to providing a laser probe beam for a Hartmann-Shack ophthalmic aberrometer. The laser probe beam produced by the system and method has a confined image spot at both the cornea and the retina. The probe beam can be generated with a laser beam passing through a moving holographic diffuser and two pinhole apertures. The holographic diffuser randomizes the spatial phase across the laser probe beam to substantially eliminate laser speckle from the Hartmann-Shack images. An imaging lens forms the probe beam and a first pinhole aperture image on the cornea, which eliminates beam size variation due to laser parameters and misalignment. A second-pinhole aperture is used to control the vergence of the probe beam and the probe beam spot size on the retina. The spot size on the retina is thus insensitive to the defocus power range of various subjects' eyes. The confined image spot on the retina substantially eliminates the possibility of an over-tight laser focal spot and allows the injection of higher laser power into the eye.

### BACKGROUND OF THE INVENTION

1. Field of the Invention

Embodiments of the invention generally relate to an ophthalmic system and method. More particularly, embodiments of the invention are directed to an ophthalmic system that provides a probe beam for optical diagnostic measurements and an ophthalmic method for generating a probe beam for optical diagnostic measurements. Embodiments of the invention are most particularly directed to apparatus and methods for making diagnostic ophthalmic wavefront measurements.

2. Description of Related Art

Ophthalmic wavefront sensors have been widely used to objectively measure higher-order aberrations of a subject's eye. The wavefront measurements are often used to provide data for customized photo-refractive surgery or other ophthalmic diagnostic and therapeutic procedures. Various types of wavefront sensors are known in the art. One of the most common types of ophthalmic wavefront sensors is the Hartmann-Shack system. In a Hartmann-Shack wavefront sensor, a probe beam is injected into the subject's eye to produce an illumination spot on the retina. The illumination light scattered from the retina exits from the eye's pupil in the form of a wavefront that is aberrated by defects in the subject's eye. The aberrated wavefront is input to a Hartmann-Shack wavefront sensor. The Hartman-Shack apparatus includes an array of lenslets that form an array of aerial images on a detector. The relative positions of the aerial images on the detector are processed to provide detailed information about the subject's vision defects.

It is desirable, when using a Hartmann-Shack ophthalmic wavefront instrument, that the light probe beam have a small beam spot size on both the anterior corneal surface and on the retina. A small beam spot size on the cornea reduces corneal reflections into the Hartmann-Shack sensor as well as minimizes aberrations due to corneal surface irregularities. A smaller beam spot on the retina enables formation of smaller, better defined Hartmann-Shack images on the detector. It is also very desirable that the beam spot size on the retina not change substantially over the range of focusing powers of different subjects' eyes.

A narrow, coherent or semi-coherent light beam is commonly employed as a probe beam for a Hartmann-Shack ophthalmic wavefront instrument. Typically a laser or a super luminescence diode (SLD) serves as the probe beam light source due to their light brightness and good beam quality. This narrow, coherent beam, typically referred to as a Gaussian beam, can relatively easily be focused into a small spot on the cornea and the retina. The lenslet arrays in state of the art Hartmann-Shack devices have individual lenslet diameters of 200μ, which can produce image spots having diameters less than 50μ. However, a coherent light beam may produce an over-tight focal spot on retina. An over-tight focal spot on retina limits the light power that can safely be injected into the eye. Moreover, it is desirable that the image spot diameter be larger than the size of a single pixel in the detector, currently about 5 to 10μ on a side. The temporal coherence of the beam also produces beam speckle, which degrades the quality of the Hartmann-Shack aerial images. The well know speckle phenomena causes localized hot spots in the focused image as opposed to a uniform, round image spot.

Various solutions have been implemented to address the problems associated with probe beam spot size on the cornea and the retina, retinal beam spot size stability over the common range of focusing power of the subject pool population, and image quality degradation due to beam speckle. For example, a Gaussian laser beam can be focused with a long focal length lens to locate the beam waist in front of or behind the retinal surface. In another technique, the laser probe beam is focused onto the cornea, which then acts as a point source for illuminating the subject's retina. While these approaches may be relatively effective for beam size confinement and focusing considerations, they do not address the speckle issue.

A further limitation with a narrow laser probe beam is that the beam size is sensitive to laser beam adjustment and variation. This limitation becomes troublesome particularly when a diode laser is used. Although a diode laser is desirable for its compactness and lower cost, it exhibits less-than-ideal beam quality and beam profile stability. Beam shape and spot size on the cornea and the retina are sensitive to diode laser alignment and collimation. Beam shape and spot size may vary as laser power changes and the laser diode ages.

Lai et al. disclose the use of a holographic diffuser to create a speckle free laser probe beam for ophthalmic wavefront measurement. A holographic phase plate is disposed in the path of a coherent light beam and scanned rapidly to randomize the spatial coherence across the beam.

A super luminescence diode (SLD) may replace a laser for a probe beam when high brightness and good beam quality, but reduced speckle is desired. The SLD probe beam has a shorter coherence length than a laser beam and produces a weaker speckle effect. However, spatial coherence across the beam from a SLD is substantially the same as that from a laser. Therefore, speckle reduction with a SLD is not complete. In addition, the selection of SLD power, wavelength, and vendors is more limited than that for lasers.

In view of the foregoing issues, problems, attempted solutions and desirable objectives, the inventors have recognized the need for an ophthalmic system and method, particularly suitable for use in measuring ocular wavefronts, that effectively address the issues and problems, provide better solutions, achieve desired objectives and which do so in a manner that is both technically and cost efficient.

### SUMMARY

An embodiment of the invention is directed to an ophthalmic system. The ophthalmic system includes a light source adapted to produce at least semi-coherent light along a source light path; a diffuser disposed in the source light path that is adapted to randomize the spatial coherence of the at least semi-coherent light and produce a diffused light output; a first aperture disposed along an optical axis in a path of the diffused light output; an optical component disposed along the optical axis that is adapted to form a probe beam as well as an image of the first aperture at a first predetermined image plane location; and a second aperture that is disposed along the optical axis adjacent the optical component, which is adapted to limit a vergence of the probe beam and to limit a probe beam spot size at a second predetermined image plane location. In this respect, the second aperture acts as a field stop and may be located proximate the optical component on the upstream or downstream side of the optical component.

In an aspect, the ophthalmic system is particularly suited to providing a diagnostic wavefront probe beam. In particular various aspects, the light source is a laser or a super luminescent diode; the diffuser is a holographic diffuser and, more particularly, a scanning, rotating or otherwise dynamic diffuser that randomizes the spatial coherence of the light to reduce or eliminate speckle; the first and second apertures are what are commonly referred to as pinhole apertures having respective diameters of between about 50 to 200μ and between about 1 to 4 mm. According to an aspect, the image of the first aperture at the first predetermined image plane location has a diameter equal to or less than about 500μ and the probe beam spot size at the second predetermined image plane location has a diameter in a range between about 70 to 130μ, and more particularly about 100μ. In a particular exemplary aspect, the first predetermined image plane location will be made to correspond to an anterior corneal surface of a test subject's eye that is operatively engaged with the ophthalmic system, and the second predetermined image plane location will then correspond to a retinal surface of a subject's eye having a nominal defocusing power of zero diopters. In this sense, the subject's eye represents a focusing optical subsystem. Known means in the form of positioning apparatus are provided for positioning the first predetermined image plane location and the second predetermined image plane location relative to an object such as a focusing optical subsystem. For example, s subject will position their head in a chin rest or a bite bar apparatus or the like. This will approximately position the subject's cornea with the first predetermined image location. The system itself can be mounted on a positioning subsystem to then fine tune the location of the first predetermined image location relative to the anterior corneal surface of the subject's eye. According to an aspect, the ophthalmic system further includes a wavefront sensor that is adapted to measure a wavefront exiting the subject's eye. In a particular aspect, the wavefront sensor is a Hartmann-Shack type wavefront sensor. In another aspect, the probe beam has an optical axis that is displaced relative to a central optical axis of the system. In a further aspect, there are no optical phase altering components along the probe beam path between the second pinhole aperture and the first predetermined image plane location if the second pinhole aperture is located optically downstream of the optical component; or, between the optical component and the first predetermined image plane location if the second pinhole aperture is located optically upstream of the optical component.

Another embodiment of the invention is directed to an ophthalmic method. The ophthalmic method includes the steps of providing an at least semi-coherent beam of light along a source light path; randomizing the spatial coherence of the at least semi-coherent beam of light to produce a diffused light output beam; illuminating a first pinhole aperture with a portion of the diffused light output beam; forming a probe beam and imaging the first pinhole aperture at a first predetermined imaging location; and illuminating a second pinhole aperture with either the diffused light output beam or the probe beam (depending on its location) to control a vergence of the probe beam and a size of the probe beam spot at a second predetermined imaging location. In an aspect, the method further comprises providing a focusing optical subsystem having an anterior surface positioned at the first predetermined imaging location and another surface that coincides with the second predetermined imaging location; and, providing a probe beam spot having a desired size at the second predetermined imaging location.

In an aspect, the ophthalmic method is particularly suited to providing a diagnostic wavefront probe beam and, further, to utilizing this wavefront probe beam for measuring a wavefront aberration of the focusing optical subsystem, which, in particular, can be a subject's eye.

In more exemplary terms, embodiments of the invention provide an apparatus and method that are used to generate a diagnostic wavefront probe beam using optical imaging to control probe beam spot shape and size on the cornea and retina of a subject's eye. A rotating, scanning or otherwise moving holographic diffuser can be used in a laser beam path to generate a diffused light source that eliminates an over-tightly focused spot on the retina. Scanning or rotating the diffuser randomizes the relative phase across the beam to minimize speckle in the Hartmann-Shack aerial images. First and second pinhole apertures are provided, which, respectively, are imaged onto the cornea and the retina to confine the size and shape of the beam spots. Further, the second pinhole aperture is used to limit the vergence of the probe beam so as to obtain a confined beam spot on the retina over a range of eye defocus powers of typically +10 D to −15 D. Embodiments of the invention are further advantageous in that the probe beam has good beam quality, high brightness, a defined wavelength, and a narrow bandwidth, similar to laser beam characteristics. In addition, the image-confined spot size and shape are not sensitive to laser misalignment and beam collimation.

The foregoing and other objects, features, and advantages of embodiments of the present invention will be apparent from the following detailed description of the embodiments, which make reference to the several drawing figures.

### BRIEF DESCRIPTION OF THE DRAWINGS

FIG. 1 is a schematic diagram of an ophthalmic system used for generating an image-confined laser probe beam according to an embodiment of the invention;

FIG. 2 is a schematic diagram of an ophthalmic system according to an exemplary aspect of the invention; and

FIG. 3 is a schematic diagram of an ophthalmic system according to another exemplary aspect of the invention.

### DETAILED DESCRIPTION OF EMBODIMENTS OF THE INVENTION

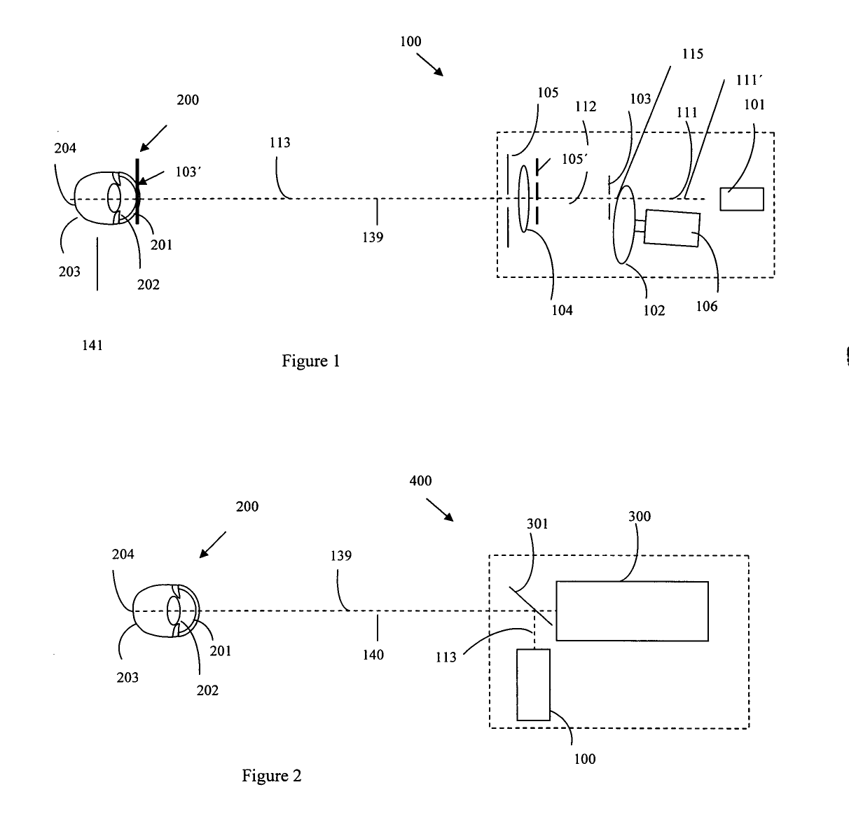

FIG. 1 is a schematic diagram of an exemplary ophthalmic system 100 that is particularly suited for generating a probe beam 113 used in diagnostic measurement of a subject's eye 200. The system 100 includes a light source 101 that produces at least a semi-coherent light beam 111 along a source light path 111′. A diffuser 102 is disposed in the source light path 111′ that produces a diffused light output 115 from the light beam 111. A first pinhole aperture 103 is disposed along an optical (eye)/instrument axis 139 in the path of the diffused light output 115. An optical component 104 is disposed along the optical axis 139. The optical component 104 forms a probe beam 113 of the diffused light output 115, as well as a first image 103′ of the first pinhole aperture 103 at a first predetermined image plane location 201. A second pinhole aperture 105 is disposed along the optical axis 139 between the optical component 104 and the first predetermined image plane location 201 and is illuminated by the probe beam 113. Means 141 are provided for locating and stabilizing a subject's eye such that the positions of the first and second image planes are precisely located relative to the eye. Exemplary means 141 include chin rests, bite bars, head stabilizers, or other well known apparatus for situating a subject's head relative to the system as well as multi-axis controllers for fine tuning the position and orientation of the system relative to the subject's head and eyes. The laser probe beam 113 is injected into a subject's eye 200 where a desired probe beam spot 204 is formed on the retina 203.

According to an exemplary aspect, the light source 101 is a laser that produces a relatively narrow laser beam 111 with a predetermined beam size, power, and wavelength. Illustratively, the beam size at the diffuser 102 will be about 0.1-0.5 mm. The laser source 101 may particularly be a diode laser module, which is advantageous due to compact size, high reliability, and low cost. Other coherent or semi-coherent light sources that provide high brightness, such as a super luminescence diode (SLD), may be used. A suitable diode laser module for the exemplary application will have an output power of 0.1-10 mW at a wavelength in the near-infrared range of between about 760 to 1000 nm.

In an exemplary aspect, the diffuser 102 will be a rotating holographic diffuser such as that disclosed in U.S. Pat. No. 6,952,435, the disclosure of which is herein incorporated by reference in its entirety to the fullest allowable extent. The holographic diffuser can be made with a fine and uniform holographic pattern embossed on a thin acrylic substrate or other suitable material. The exemplary holographic diffuser 102 will particularly have a small but well-defined diffusing angle in the range of about 0.5 to 5 degrees. It may be desirable to focus the source light on the diffuser. A motor 106 is connected to the diffuser 102 to rotate or otherwise scan the diffuser across the laser beam 111. This serves to randomize the relative phase across the laser beam and minimize or eliminate speckle due to coherence effects.

The first pinhole aperture 103 is located along the system optical axis 139 immediately optically downstream of the holographic diffuser 102. It is illuminated with the diffused laser light output 115. The exemplary first pinhole aperture 103 has a circular diameter between about 50 to 200 micron.

As illustrated, the optical component 104 is a focal lens that refocuses the light 112 transmitted through the first pinhole aperture 103 into a probe beam 113. The focal lens 104 is positioned, and has optical parameters, such that it forms an image 103′ of the first pinhole aperture 103 onto a first predetermined image plane, which in the illustrative embodiment is the anterior corneal surface 201 of the subject's eye. The probe beam spot on the cornea 201 is thus confined by the image size 103′ of the first pinhole aperture 103. In the exemplary embodiment, the focal lens 104 has a focal length of between about 30 to 100 mm. The working distance from the focal lens 104 to the first image plane 201 is between about 150 to 300 mm. Other focal optical components may be used to perform the desired function, and may include diffractive or holographic components, for example.

The second pinhole aperture 105 is located adjacent the front surface of the focal lens 104 and is illuminated by the probe beam 113 formed by the focal lens. Alternatively, the second pinhole aperture could be located adjacent the rear surface of the focal lens 104 as shown at 105′ and be illuminated by the diffused light output 112. In the exemplary embodiment, the second pinhole aperture has a circular diameter between about one (1) to four (4) mm, which limits the vergence of the laser probe beam 113 propagating to the subject's eye 200. A small vergence of the laser probe beam 113, advantageously being equal to or less than about five (5) milliradians, minimizes the beam size change around the focal plane. Thus the size of the probe beam spot 204 on the retina 203 remains substantially the same for subjects' eyes with various defocusing powers over a range of about 25 diopters between about +10 to −15 diopters. It will be appreciated that in such embodiments aperture 105, 105′ and lens 104 are configured and arranged such that the size of the probe beam spot 204 remains substantially the same when used with such subjects. The term “substantially the same spot size” means that the spot does not vary by more than 50% in diameter.

A person skilled in the art will appreciate that the second pinhole aperture 105 is more or less imaged as the probe beam spot 204 onto the second predetermined image plane; that is, the retina 203, via the eye's optics. As such, the laser probe beam 113 has a beam spot 204 confined by the image size of the second pinhole aperture 105. The probe beam spot size 204 at the second predetermined image plane location 203 advantageously will have a diameter in a range between about 70 to 130μ and, more advantageously, a diameter of about 100μ. Since the laser probe beam 113 is a diffused laser beam, the beam spot 204 on the retina 203 will not have an over-tight focus as that term is known in the art. In the illustrative embodiment, the eye, comprising the cornea, a natural or artificial lens, and the retina, represents a focusing optical subsystem. The cornea (the first predetermined image plane) can be considered to be about 20 mm in front of the retina (the second predetermined image plane).

According to an exemplary embodiment described with reference to FIG. 1, the laser source 101 is a diode laser module operated at 780 nm. The laser source 101 produces a narrow laser beam 111 having a beam size of about 100μ on the holographic diffuser 102. A first pinhole aperture 103 has a 100μ diameter and is placed close to the holographic diffuser 102 to receive the diffused output beam 115. The light 112 transmitted through the first pinhole aperture 103 has a full divergence angle of about two (2) degrees and has a spot size of about three (3) mm on the focal lens 104, which is located about 80 mm from the first pinhole aperture 103. The focal lens 104 has a focal length of 60 mm and images the probe beam 113 to a spot 103′ of about 300μ on the subject's cornea 201 located about 240 mm from the focal lens 104. The second pinhole aperture 105 is located adjacent the focal lens 104 and has a diameter of 1.2 mm. The probe beam 113 thus has a vergence of about five (5) mR. Based on typical eye optical parameters, the second pinhole aperture image spot 204 (i.e., the probe beam spot at the second predetermined image plane) has a diameter of about 100μ on the retina 203. The spot size on the retina will change by less than 50% for subjects' eyes having defocusing power ranging from about −15 D to +15 D.

In a further aspect according to the instant embodiment, as illustrated in FIG. 2, the ophthalmic system depicted at 400 includes a wavefront sensor 300 that is operatively and optically connected with the aforementioned ophthalmic system 100 by a beam splitter 301. In an exemplary aspect, the wavefront sensor 300 is a Hartmann-Shack apparatus. The construction and operation of a Hartmann-Shack wavefront sensor is well known in the art and needs no further description here for a clear understanding of the invention. It will be appreciated, in any event, that high quality lenslet arrays having lenslet diameters of 200% are available. A lenslet aerial image on a detector should have a spot diameter of less than about 50μ but larger than the size of a single detector pixel (e.g., about 5-10μ on a side). The ratio of the lenslet image spot size to the probe beam spot size on the retina is directly proportional to the ratio of the lenslet focal length to the eye focal length. It will be further appreciated that embodiments of the invention are not limited to the use of a Hartmann-Shack wavefront sensor. Various other well known wavefront sensing apparatus and techniques may be suitable.

As further shown in FIG. 2, a laser probe beam 113 is generated by ophthalmic system 100. The laser probe beam 113 is directed into a subject's eye 200, via only the beam splitter 301, along a probe beam propagation axis 140 that is coincident with the optical (eye)/wavefront sensor axis 139. No optical phase altering components intercept the probe beam between the system 100 output and the corneal surface 201 of the eye. This is herein referred to as “direct injection” of the probe beam. The probe beam spot 204 on the retina 203 is scattered by the retinal surface and passes out through the cornea along the optical (eye)/wavefront sensor axis 139, through the beam splitter 301, and into the wavefront sensor 300. The wavefront sensor can then measure the wavefront aberrations caused by the eye's defects.

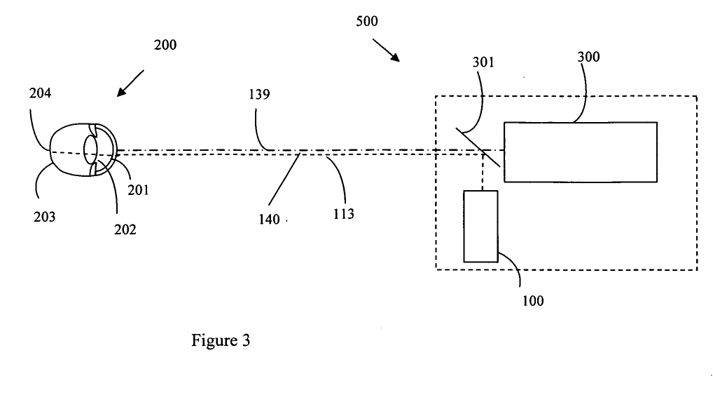

FIG. 3 is a schematic diagram of another aspect of the embodiment described with respect to FIG. 2. The system 500 in FIG. 3 differs from the system 400 in FIG. 2 only in that the propagation axis 140 of the probe beam 113 is parallely displaced from the optical (eye)/wavefront sensor axis 139 by a known amount. This is herein referred to as “off-axis” injection of the probe beam. A recognized advantage of off-axis injection is that the direct corneal reflection of the probe beam 113 is deflected away from the wavefront sensor 300. A detailed description of off-axis injection is disclosed in U.S. Pat. No. 6,264,328, the disclosure of which is herein incorporated by reference in its entirety to the fullest allowable extent.

Another embodiment of the invention is directed to an ophthalmic method. According to an aspect, the ophthalmic method is particularly suited to providing a diagnostic wavefront probe beam and, further, to utilizing this wavefront probe beam for measuring a wavefront aberration of a subject's eye. The ophthalmic method includes the steps of providing an at least semi-coherent beam of light along a source light path; randomizing the spatial coherence of the at least semi-coherent beam of light to produce a diffused light output beam; illuminating a first pinhole aperture with a portion of the diffused light output beam; forming a probe beam from the diffused light output beam and imaging the first pinhole aperture at a first predetermined imaging location; and illuminating a second pinhole aperture with either the diffused light output beam or the probe beam, depending upon its placement, to control a vergence of the probe beam and a size of the probe beam spot at a second predetermined imaging location. The method may further include the step of providing a focusing optical subsystem having an anterior surface that can be positioned at the first predetermined imaging location and another surface that will coincide with the second predetermined imaging location. According to an exemplary aspect, a subject's eye is provided as the focusing optical subsystem in which the anterior corneal surface is the surface positioned to coincide with the first predetermined imaging location, and the retinal surface of the eye is the other surface that will coincide with the second predetermined imaging location.

In conjunction with the system embodiments described above, the various method steps can be carried out in the following various exemplary manners. A laser (laser diode) or a super luminescent diode can be used for providing the at least semi-coherent beam of light. A scanning or rotating holographic diffuser can be used for diffusing the at least semi-coherent beam of light. A focusing lens can be used for imaging the first pinhole aperture at the first predetermined imaging location. As noted above, a properly positioned and stabilized eye will provide the focusing optical subsystem in which the anterior corneal surface becomes the first predetermined imaging plane and the retina is the second predetermined imaging plane. According to the method, the characteristics of the second pinhole aperture are used to control a vergence of the probe beam as it propagates towards the eye and a size of the probe beam spot at the second predetermined imaging location. A probe beam image spot (first pinhole aperture image) has a diameter equal to or less than about 500μ on the anterior corneal surface, and the probe beam spot formed on the retinal surface has a diameter in a range between about 70 to 130μ. A wavefront sensor and, in particular, a Hartmann-Shack apparatus can be used to measure wavefront aberration of the subject's eye. The probe beam can be directly injected into the subject's eye. The probe beam can also be injected off-axis into the subject's eye along a probe beam propagation axis that is displaced relative to an optical/instrument axis.

The foregoing description of the preferred embodiments of the invention have been presented for the purposes of illustration and description. It is not intended to be exhaustive or to limit the invention to the precise forms disclosed. Many modifications and variations are possible in light of the above teachings. It is intended that the scope of the invention embodiments be limited not by this detailed description but rather by the claims appended hereto.

We claim:

1. A ophthalmic system, comprising:
a light source adapted to produce at least semi-coherent light along a source light path;
a diffuser disposed in the source light path adapted to produce a randomized spatially coherent light output from the at least semi-coherent light;
a first aperture disposed along an optical axis in a path of the diffused light output;
a focal optical component disposed along the optical axis, wherein the optical component is adapted to form a probe beam, further wherein the optical component is adapted to form an image of the first aperture at a first predetermined image plane location; and
a second aperture disposed along the optical axis adjacent the optical component.

2. The system of claim 1, wherein the light source is a laser.

3. The system of claim 1, wherein the light source is a super luminescent diode.

4. The system of claim 1, wherein the diffuser is a holographic diffuser.

5. The system of claim 4, wherein the diffuser is one of a scanning and a rotating diffuser.

6. The system of claim 4, wherein the diffuser has a diffusing angle between about 0.5 to 5.0 degrees.

7. The system of claim 1, wherein a source light spot incident on the diffuser has a spot size diameter between about 0.1 to 0.5 mm.

8. The system of claim 1, wherein the second aperture is located intermediate the optical component and the first predetermined image plane location.

9. The system of claim 1, wherein the first aperture has a diameter of between about 50 to 200 micrometers (μ).

10. The system of claim 1, wherein the second aperture has a diameter of between about 1 to 4 millimeters (mm).

11. The system of claim 1, wherein the optical component is a focal lens having a focal length in the range between about 30 to 100 mm.

12. The system of claim 1, wherein the probe beam exiting the second aperture has a vergence equal to or less than 5 milliradians (mR).

13. The system of claim 1, wherein the image of the first aperture at the first predetermined image plane location has a diameter equal to or less than about 500μ.

14. The system of claim 1, further comprising means for positioning the first predetermined image plane location relative to an object.

15. The system of claim 14, wherein the object is a focusing optical subsystem, further wherein the first predetermined image plane location coincides with an anterior surface of the focusing optical subsystem.

16. The system of claim 15, wherein the focusing optical subsystem is the ocular system of a test subject, further wherein the anterior surface of the focusing optical subsystem is the anterior corneal surface of the ocular system.

17. The system of claim 1, wherein the probe beam spot size at a second predetermined image plane location has a diameter in a range between about 70 to 130μ.

18. The system of claim 17, wherein the probe beam spot size at the second predetermined image plane location has a diameter of about 100μ.

19. The system of claim 15, further comprising a wavefront sensor adapted to measure a wavefront exiting the focusing optical subsystem.

20. The system of claim 19, wherein the wavefront sensor is a Hartmann-Shack type wavefront sensor.

21. An ophthalmic system, comprising:
a light source adapted to produce at least a semi-coherent light beam along a source light path;
a diffuser disposed in the source light path adapted to produce a randomized spatially coherent light output from the light beam;
a first pinhole aperture disposed along an optical axis in a path of the diffused light output;
a focal optical component disposed along the optical axis, wherein the optical component is adapted to form a probe beam of the diffused light output, further wherein the optical component is adapted to form a first image of the first aperture at a first predetermined image plane location;
a second pinhole aperture disposed along the optical axis adjacent the optical component, wherein the second aperture is adapted to provide a controlled vergence of the probe beam and a probe beam spot at a second predetermined image plane location; and,
a positioning apparatus capable of locating a subject's eye relative to the first predetermined image plane location and the second predetermined image plane in a path of the probe beam.

22. The ophthalmic system of claim 21, wherein the first predetermined image plane location is adapted to coincide with an anterior corneal surface of a subject's eye that is operatively engaged with the ophthalmic system, and the second predetermined image plane location is adapted to coincide with a retinal surface of a subject's eye.

23. The ophthalmic system of claim 21, wherein the light source is a laser.

24. The ophthalmic system of claim 21, wherein the light source is super luminescent diode.

25. The ophthalmic system of claim 21, wherein the light source has a wavelength in a range between about 780 to 1000 nanometers (nm), and a power level between about 0.1 to 10 milliwatts (mW).

26. The ophthalmic system of claim 21, wherein the diffuser is a holographic diffuser.

27. The ophthalmic system of claim 26, wherein the diffuser is a scanning holographic diffuser.

28. The ophthalmic system of claim 26, wherein the diffuser is a rotating holographic diffuser.

29. The ophthalmic system of claim 21, wherein the diffuser has a diffusing angle between about 0.5 to 5.0 degrees.

30. The system of claim 21, wherein a source light spot incident on the diffuser has a spot size diameter between about 0.1 to 0.5 mm.

31. The system of claim 21, wherein the first pinhole aperture has a diameter of between about 50 to 200μ.

32. The system of claim 21, wherein the second pinhole aperture has a diameter of between about 1 to 4 mm.

33. The system of claim 21, wherein the second pinhole aperture is located intermediate the optical component and the first predetermined image plane location.

34. The system of claim 21, wherein the optical component is a focal lens having a focal length in the range between about 30 to 100 mm.

35. The system of claim 21, wherein the probe beam exiting the second aperture has a vergence equal to or less than 5 mR.

36. The system of claim 22, wherein the image of the first aperture on the subject's cornea has a diameter equal to or less than about 500μ.

37. The system of claim 36, wherein the probe beam spot on the subject's retina has a diameter in a range between about 70 to 130μ.

38. The system of claim 37, wherein the probe beam spot has a diameter of about 100μ.

39. The system of claim 22, further comprising a wavefront sensor adapted to measure a wavefront aberration of the subject's eye.

40. The system of claim 39, wherein the wavefront sensor is a Hartmann-Shack type wavefront sensor.

41. The system of claim 21, wherein the probe beam has an optical axis that is displaced relative to a central optical axis of the system.

42. The system of claim 21, wherein there are no optical phase altering components along the probe beam path intermediate the second pinhole aperture and the anterior corneal surface of the subject's eye.

43. A ophthalmic method, comprising:
providing an at least semi-coherent beam of light along a source light path;
randomizing the spatial coherence of the at least semi-coherent beam of light to produce a diffused light output beam;
illuminating a first pinhole aperture with a portion of the diffused light output beam;
forming a probe beam from the diffused light output beam and imaging the first pinhole aperture at a first predetermined imaging location;
illuminating a second pinhole aperture with one of the diffused light output beam and the probe beam to control a vergence of the probe beam and a size of the probe beam spot at a second predetermined imaging location.

44. The method of claim 43, further comprising providing a focusing optical subsystem having an anterior surface positioned at the first predetermined imaging location and another surface that coincides with the second predetermined imaging location.

45. The method of claim 43, comprising utilizing a laser for providing the at least semi-coherent beam of light.

46. The method of claim 43, comprising utilizing a super luminescent diode for providing the at least semi-coherent beam of light.

47. The method of claim 43, comprising utilizing one of a scanning holographic diffuser and a rotating holographic diffuser for randomizing the spatial coherence of the at least semi-coherent beam of light.

48. The method of claim 43, comprising utilizing a focusing lens for imaging the first pinhole aperture at the first predetermined imaging location.

49. The method of claim 44, comprising providing a subject's eye as the focusing optical subsystem, wherein an anterior corneal surface is the first predetermined imaging location and a retinal surface is the second predetermined imaging location.

50. The method of claim 49, comprising forming an image spot having a diameter equal to or less than about 500μ on the anterior corneal surface, and forming the probe beam spot having a diameter in a range between about 70 to 130μ on the retinal surface.

51. The method of claim 50, comprising measuring a wavefront aberration of the subject's eye.

52. The method of claim 51, comprising directly injecting the probe beam into the subject's eye.

53. The method of claim 51, comprising injecting the probe beam into the subject's eye along a probe beam propagation axis that is displaced relative to an optical/instrument axis.

54. A ophthalmic system, comprising:
a light source adapted to produce at least semi-coherent light along a source light path;
a diffuser disposed in the source light path adapted to produce a randomized spatially coherent light output from the at least semi-coherent light;
a first aperture disposed along an optical axis in a path of the diffused light output;
a focal optical component disposed along the optical axis, wherein the optical component is adapted to form a probe beam, further wherein the optical component is adapted to form an image of the first aperture at a first predetermined image plane location; and
a second aperture disposed along the optical axis and positioned relative to the focal optical component such that, when the probe beam is directed onto patients' eyes, the eyes having defocusing powers in the range of +10 to −15 diopters, a spot diameter on the patients' retinas does not vary by more than 50%.
